(19) United States
(12) Patent Application Publication
(10) Pub. No.: US 2023/0238142 A1
(43) Pub. Date: Jul. 27, 2023

(54) SYSTEMS AND METHODS FOR IDENTIFYING INDIVIDUALS WITH A SLEEPING DISORDER AND A DISPOSITION FOR TREATMENT

(71) Applicant: ResMed Pty Ltd, Bella Vista NSW (AU)

(72) Inventor: Natalie Zotelo, San Diego, CA (US)

(21) Appl. No.: 18/001,631

(22) PCT Filed: Jun. 26, 2021
(86) PCT No.: PCT/IB2021/055727

§ 371 Date: Dec. 13, 2022

Related U.S. Application Data
(60) Provisional application No. 63/045,397, filed on Jun. 29, 2020.

(51) Int. Cl. G16H 50/30; G16H 10/60; G16H 20/00; G16H 50/70; G16H 80/00

(52) U.S. Cl. CPC G16H 50/30; G16H 10/60; G16H 20/00; G16H 50/70; G16H 80/00

(57) ABSTRACT

A system and method includes (i) providing patient data stored in a data repository, (ii) applying a first patient identification algorithm to the patient data to identify an initial group of individuals associated with select physical and health characteristics, (iii) applying a second patient identification algorithm to the patient data associated with the initial group of individuals to identify a narrower subgroup associated with select behavioral characteristics, and (iv) generating patient identifiable information from the patient data to allow for notification. The identification of the initial group is based on a determined likelihood of obstructive sleep apnea (OSA) for individuals meeting or exceeding a first threshold criteria. The identification of the narrower group is based on a determined likelihood of long-term adherence to OSA treatment for individuals meeting or exceeding a second threshold criteria. The notification is of designated entities that one or more of the individuals in the narrower subgroup are preferred individuals for OSA.

### CROSS-REFERENCE TO RELATED APPLICATIONS

This application claims priority to, and the benefit of, U.S. Provisional Pat. Application No. 63/045,397, filed on Jun. 29, 2020, the disclosure of which is hereby incorporated by reference herein in its entirety.

### TECHNICAL FIELD

The present disclosure relates generally to systems and methods for identifying individuals with certain physical and health characteristics suggestive of obstructive sleep apnea; and more specifically, the present disclosure relates systems and methods that further identify individuals with behavioral characteristics suggesting long-term adherence to obstructive sleep apnea treatment.

### BACKGROUND

Many individuals suffer from sleep-related and/or respiratory-related disorders such as, for example, Periodic Limb Movement Disorder (PLMD), Restless Leg Syndrome (RLS), Sleep-Disordered Breathing (SDB) such as Obstructive Sleep Apnea (OSA), Central Sleep Apnea (CSA), other types of apneas such as mixed apneas and hypopneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, insomnia, and chest wall disorders. These disorders are often treated using a respiratory therapy system.

However, some users find such systems to be uncomfortable, difficult to use, expensive, aesthetically unappealing and/or fail to perceive the benefits associated with using the system. As a result, some users will elect not to begin using the respiratory therapy system or discontinue use of the respiratory therapy system absent a demonstration of the severity of their symptoms when respiratory therapy treatment is not used. As a result, some users will discontinue use of the respiratory therapy system absent encouragement or affirmation that the respiratory therapy system is improving their sleep quality and reducing the symptoms of these disorders. The present disclosure is directed to solutions to these and other problems.

### SUMMARY

According to some implementations of the present disclosure, a method includes providing patient data stored in a data repository. The patient data includes physical, health, and behavioral data corresponding to identifiable individuals. The method also includes applying a first patient identification algorithm to process at least a portion of the patient data to identify an initial group of individuals associated with select physical and health characteristics. The identification of the initial group of individuals is based on a determined likelihood of obstructive sleep apnea for identifiable individuals meeting or exceeding a first threshold criteria. The method also includes applying a second patient identification algorithm to process at least a portion of the patient data associated with the initial group of individuals to identify a narrower subgroup of individuals associated with select behavioral characteristics. The identification of the narrower group of individuals is based on a determined likelihood of long-term adherence to obstructive sleep apnea treatment for individuals in the narrower subgroup meeting or exceeding a second threshold criteria. Patient identifiable information is generated from the patient data to allow for notification of one or more designated entities that one or more of the individuals in the narrower subgroup are preferred individuals for obstructive sleep apnea treatment.

According to some implementations of the present disclosure, a system includes a control system including one or more processors and a memory having stored thereon machine readable instructions. The control system is coupled to the memory, and the method is implemented when the machine executable instructions in the memory are executed by at least one of the one or more processors of the control system.

According to some implementations of the present disclosure, a system identifies individuals likely to have a potential sleeping disorder and likely to adhere to a prescribed long-term treatment plan. The system includes a control system a control system configured to implement the method.

According to come implementation, a computer program product includes instructions which, when executed by a computer, cause the computer to carry out the method.

According to come implementations, the computer program product is a non-transitory computer readable medium.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1A is a functional block diagram of an exemplary system for analyzing data to identify individuals with sleeping disorders and having a long-term disposition to adopt a sleep disorder treatment plan, according to some implementations of the present disclosure.

FIG. 1B is a functional block diagram of another exemplary system for analyzing data to identify individuals with sleeping disorders and having a long-term disposition to adopt a sleep disorder treatment plan, according to some implementations of the present disclosure.

FIG. 2 is a process flow diagram of an exemplary method for identifying individuals with sleeping disorders and having a long-term disposition to adopt a sleep disorder treatment plan, according to some implementations of the present disclosure.

FIG. 3 is a process flow diagram of an exemplary method for training algorithms for identifying individuals with sleeping disorders and having a long-term disposition to adopt a sleep disorder treatment plan, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB) such as Obstructive Sleep Apnea (OSA), Central Sleep Apnea (CSA), and other types of apneas such as mixed apneas and hypopneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hyper tension, diabetes, stroke, insomnia, and chest wall disorders.

Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate and posterior oropharyngeal wall. These disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

Obstructive Sleep Apnea (OSA) causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Respiratory pressure therapy (RPT) devices may be used individually or as part of a system to deliver one or more of a number of therapies, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include Continuous Positive Airway Pressure (CPAP) devices.

CPAP therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance. Treatment of OSA by CPAP therapy may be voluntary, and hence patients may elect not to comply with therapy if they find devices used to provide such therapy one or more of: uncomfortable, difficult to use, expensive and aesthetically unappealing.

Not all respiratory therapies aim to deliver a prescribed therapy pressure. Some respiratory therapies aim to deliver a prescribed respiratory volume, possibly by targeting a flow rate profile over a targeted duration. In other cases, the interface to the patient’s airways is ‘open’ (unsealed) and the respiratory therapy may only supplement the patient’s own spontaneous breathing. In one example, High Flow therapy (HFT) is the provision of a continuous, heated, humidified flow of air to an entrance to the airway through an unsealed or open patient interface at a “treatment flow rate” that is held approximately constant throughout the respiratory cycle. The treatment flow rate is nominally set to exceed the patient’s peak inspiratory flow rate. HFT has been used to treat OSA, CSR, COPD and other respiratory disorders. One mechanism of action is that the high flow rate of air at the airway entrance improves ventilation efficiency by flushing, or washing out, expired CO2 from the patient’s anatomical dead space. HFT is thus sometimes referred to as a dead space therapy (DST). In other flow therapies, the treatment flow rate may follow a profile that varies over the respiratory cycle.

A wide variety of physical and health characteristics of an individual can be attributable to, or be intensified by, OSA. For example, physical characteristics directly or indirectly attributable to, or intensifying, OSA can include an individual’s neck circumference, weight, gender, blood pressure, age, body mass index, and other characteristics. Health characteristics directly or indirectly attributable to, or intensified by, OSA can include snoring history, heart conditions, history of tiredness, observed apnea, diabetes, and other characteristics. Furthermore, certain behavioral characteristics of an individual having or likely to have OSA, and likely to comply with a long-term treatment plan for OSA, include an individual’s demographic information, such as education, employment, place of residence, marital status, and others. Additional behavioral characteristics of an individual having, or likely to have, OSA, and likely to comply with a long-term treatment plan for OSA, can include an individual’s motivation, fitness level, exercise routine, adherence to prescribed medication protocols, adherence to prior doctor recommendations, and other characteristics.

The data associated with the physical, health, and behavioral characteristics of an individual are collected by various sources and can be stored as historical patient data, which may be a part of a healthcare record. The data may be collected by healthcare providers during patient visits, and stored, for example within a care management platform. Data may also be collected by integrated delivery networks, healthcare systems, health care payors, and other administrators. In some instances, the data may be provided directly or indirectly by a patient. In some instances, the data may be collected by the doctor or other healthcare professional. In yet other instances, data such as behavioral information, may be collected from third party sources to the extent such data can be attributable to an individual’s behavioral, physical, and health characteristic data. All this data can be stored in a data repository. A desirable implementation of the systems and methods of the present disclosure is to identify individuals from a data repository who have certain physical and health characteristics suggestive of obstructive sleep apnea, and to further identify individuals who have certain behavioral characteristics suggesting long-term adherence to OSA treatment.

OSA is a contributing factor to many other medical issues that increase long-term expenses for health care providers and payors, along with having a profound impact on the quality of life of an individual with OSA. Where a patent with the medical issue is determined to have OSA, treating the OSA condition can minimize, or in some instances eliminate, the medical issue. This can be desirable as long-term healthcare expenses are minimized and the individual’s quality of life increases, especially where OSA is treated early. OSA has many positive benefits, but not all individuals that have been prescribed an OSA treatment plan adhere to the treatment in the long term, which can reduce the treatment benefits. A desirable aspect of the present disclosure is the identification of individuals from a repository of historical patient data that are likely to adhere to an OSA treatment plan that are initially identified as likely to have OSA based on their physical and health characteristic data.

A system is contemplated that receives or has access to data from a database, such as a database of patient health records, and uses a first trained algorithm to identify current patients that are likely to have OSA to generate an initial group of individuals. Then some or all of the data for each of the individuals in the initial group is processed through a second trained algorithm to identify current patients that are likely to adopt and/or adhere long term to an OSA treatment therapy (e.g., CPAP, mandibular repositioning device, stimulation therapy, life style changes) to generate a subgroup of the initial group of individuals. In some aspects, the subgroup of individuals is the main output of the contemplated system and may have patient identifiable information associated with each of the individuals in the subgroup. This subgroup of individuals can then be identified to a healthcare provider, healthcare payor, or to the individual themselves, as candidates that should be consulted about OSA treatment and an expected benefit of minimizing long-term healthcare expenses and increasing quality of life.

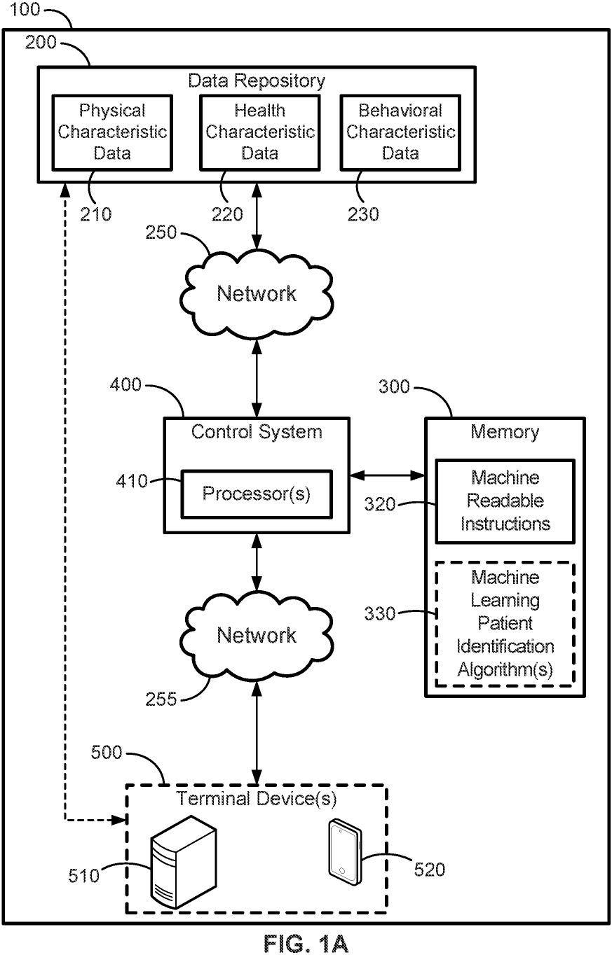

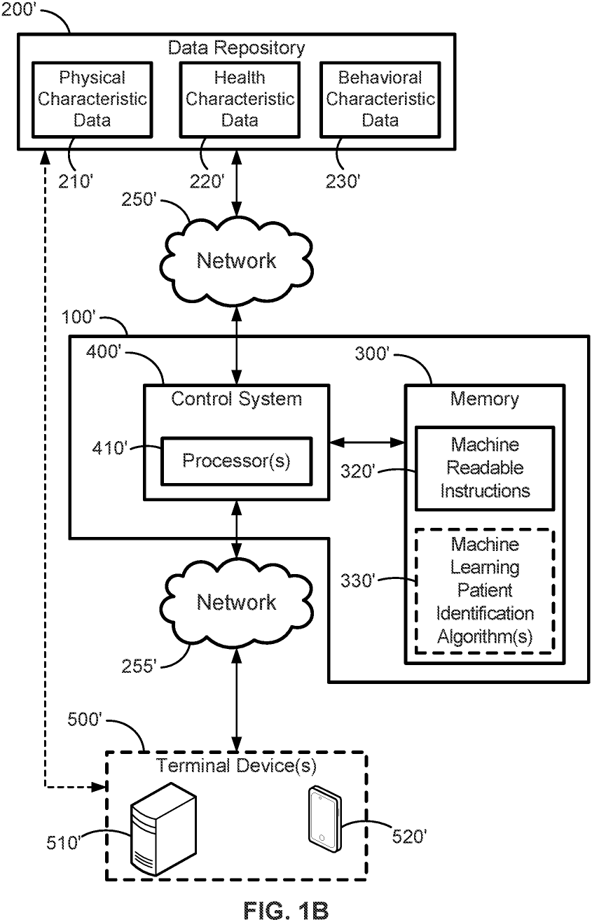

Referring to FIGS. 1A and 1B, a system 100, 100′ includes a data repository 200, 200′, a memory 300, 300′, a control system 400, 400′, and one or more terminal devices 500, 500′ (hereinafter, terminal device 500, 500′). As described herein, the systems 100, 100′ generally can be used for identifying individuals (e.g., patients of a healthcare provider) likely to have a potential sleeping disorder (e.g., obstructive sleep apnea) and that are likely to adhere to a prescribed long-term treatment plan (e.g., by a doctor or other prescriber).

While the systems 100, 100′ are shown as including various elements, the systems 100, 100′ can include any portion and/or subset of the elements shown and described herein and/or the systems 100, 100′ can include one or more additional elements not specifically shown in FIGS. 1A or 1B. Data repositories 200, 200′ are communicatively coupled to respective networks 250, 250′. In some implementations, data repositories 200, 200′ are communicatively connected via their respective networks 250, 250′, or via another network 255, 255′, to respective control systems 400, 400′ and/or to one or more respective terminal devices 500, 500′.

Data repositories 200, 200′ include a plurality of storage devices for storing patient or patient attributable data. In some implementations of the present disclosure, the data repositories 200 and 200′ can include electronic health data records for individuals and may have physical characteristic data 210 (or 210′ in FIG. 1B) for a plurality of individuals, along with health characteristic data 220 (or 220′ in FIG. 1B) and behavioral characteristic data 230 (or 230′ in FIG. 1B). While data repositories 200 and 200′ (in FIG. 1B) are shown as including various storage devices, the data repository 200 or 210′ can include any subset of the elements shown and described herein and/or the data repository 200 or 210′ can include one or more additional elements not specifically shown in FIGS. 1.

The data stored in the data repository 200 or 200′ (in FIG. 1B) can include a wide variety of types and/or contents of data. For example, in some implementations, the data stored in the data repository 200 or 200′ include physical characteristic data directly or indirectly attributable to, or intensifying, OSA such as neck circumference, weight, gender, blood pressure, age, and/or body mass index. In another example, the data include health characteristic data directly or indirectly attributable to, or intensifying, OSA such as snoring history, heart conditions, history of tiredness, observed apnea, and/or diabetes. In another example, the data include certain behavioral characteristics of an individual having or likely to have OSA, and likely to comply with a long-term treatment plan for OSA, such as demographic information, such as education, employment, place of residence, marital status, and/or healthcare payor information. In some implementations, the data include additional behavioral characteristic data of an individual having, or likely to have, OSA, and likely to comply with a long-term treatment plan for OSA, such as motivation, fitness level, exercise routine, adherence to prescribed medication protocols, and/or adherence to prior doctor recommendations. The data stored in data repository 200 or 200′ include historical patient data, such as the physical, health, and behavioral characteristic data, that correspond to identifiable individuals (e.g., current or former patients).

Additional data stored in the data repository 200 or 200′ that correspond to identifiable individuals are further detailed. For another example, in some implementations, the data includes adherence data associated with multiple individuals that are similar to the individual. For another example, in some implementations, the data includes a determination of whether the individual encounters difficulties breathing during sleep. For another example, in some implementations, the data includes relationship information of the individual. For another example, in some implementations, the data includes web searches performed by the individual. For another example, in some implementations, the data includes a determination of whether the individual is likely to exhibit binge-like behavior, a determination of whether the individual is likely to change behavior, or both. For another example, in some implementations, the data includes a summary of at least a portion of a historical account of clinical behavior that the individual has changed. For another example, in some implementations, the data includes one or more daily health assessments that include the occurrence and/or frequency of headaches and/or migraines experiences by the individual. For another example, in some implementations, the data includes dependent-family information of the individual. For another example, in some implementations, the data includes subscriptions of the individual in mobile-based or web-based health applications, social media information associated with the individual, or any combination thereof. For another example, in some implementations, the data includes a determination of a tendency of the individual to be an early adopter of technology. For another example, in some implementations, the data includes information associated with whether the individual is a drug user, information associated with whether the individual consumes alcohol, or any combination thereof. For another example, in some implementations, the data includes information such as age, gender, BMI, health information, whether the individual is a smoker or a non-smoker, whether the individual drinks alcohol, or any combination thereof. For another example, in some implementations, the data includes information such as self-reported pain points such as daytime drowsiness, snoring, fatigue, exercise level (duration, intensity, type), difficulties staying asleep, etc., or any combination thereof. It is understood the data stored in the data repository 200 or 210′ can include any combination of the above described types of data and/or other types of data not specifically described herein.

In some implementations, the control system 400 (or 400′ in FIG. 1B) executes machine-readable instructions (stored in respective memory 300 in FIG. 1A or 300′ in FIG. 1B, or a different memory or in both) to apply a first patient identification algorithm to process at least a portion of the patient data to identify an initial group of individuals associated with select physical and health characteristics. The identification of the initial group of individuals is based on a determined likelihood of obstructive sleep apnea for identifiable individuals meeting or exceeding a first threshold criteria or predetermine threshold value. The control system 400 or 400′ further executes machine-readable instructions (stored in respective memory 300 or 300′, or a different memory or in both) to apply a second patient identification algorithm to process at least a portion of the patient data associated with the initial group of individuals to identify a narrower subgroup of individuals associated with select behavioral characteristics. The identification of the narrower group of individuals is based on a determined likelihood of long-term adherence to obstructive sleep apnea treatment for individuals in the narrower subgroup meeting or exceeding a second threshold criteria or predetermined threshold value. Finally, the control system 400 or 400′ executes machine-readable instructions (stored in respective memory 300 or 300′, or a different memory or in both) to generating patient identifiable information from the patient data to allow for notification of one or more designated entities that one or more of the individuals in the narrower subgroup are preferred individuals for obstructive sleep apnea treatment. In some implementations, the patient identification algorithms may be machine learning algorithms. In some implementations, the patient identification algorithms may be pre-programmed algorithms. In some implementations, the preprogrammed algorithms can be updated at predetermined intervals as desired by a user.

In some implementations, the data stored in the data repository 200 or 200′ can include training data (e.g., historical, real-time) that is associated with a plurality of individuals. In some such implementations, the control system 400 or 400′ executes machine-readable instructions (stored in respective memory 300 or 300′, or a different memory or in both) to train a machine learning patient identification algorithm(s) 330 in FIG. 1A or 330′ in FIG. 1B (stored in the memory 300 or 300′, or a different memory or in both) with the training data. By using the training data, machine learning patient identification algorithm(s) 330 or 330′ are configured to receive as an input at least a portion of the data stored in the data repository 200 or 200′ that are associated with identifiable individuals.

The one or more terminal devices 500 in FIG. 1A or 500′ in FIG. 1B can be associated with individuals, a healthcare provider, an integrated delivery network, a healthcare payor, an administrator, or another designated entity. In some implementations, the terminal devices 500 (or 500′) are configured to receive one or more notifications from the control system 400 or 400′. In some implementations, the notification includes that one or more of the individuals in a narrower subgroup, as identified by the patient identification algorithms, are preferred (e.g., likely to adhere to long-term treatment) individuals for OSA treatment. The one or more terminal devices 500 or 500′ can include a personal computer 510 (or 510′ in FIG. 1B), a mobile device 520 (or 520′ in FIG. 1B), or any combination thereof. In some implementations, the terminal device 500 or 500′ can communicate data to and/or receive data from the data repository 200 or 200′, such as patient data that might be sent to the data repository whether as part of the health care record or data received directly from an individual (e.g., a patient).

In some implementations, the memory 300 or 300′ stores the machine-readable instructions 320 or 320′ and the first and second patient identification algorithms. The control system 400 or 400′ is communicatively coupled to respective memory 300 or 300′ and includes one or more processors 410 or 410′. The control system 400 is generally used to control (e.g., actuate) the various components of the system 100 and/or analyze data obtained and/or generated by the components of the system 100. The control system 400′ is similarly used to control (e.g., actuate) the various components of the system 100′ and/or analyze data obtained and/or generated outside the system by the components 200′ and/or 500′. The processor 410 (or 410′ in FIG. 1B) executes respective machine readable instructions 320 (or 320′ in FIG. 1B) that are stored in the respective memory device 300 or 300′ and can be a general or special purpose processor or microprocessor.

While one processor 410 is shown in FIG. 1A and one processor in FIG. 1B, the respective control system 400 or 400′ can include any suitable number of processors (e.g., one processor, two processors, five processors, ten processors, etc.). The respective memory 300 or 300′ can be any suitable computer readable storage device or media, such as, for example, a random or serial access memory device, a hard drive, a solid state drive, a flash memory device, etc. The control system 400 and/or the memory 300 can be coupled to and/or positioned within a housing of one or more of the terminal devices 500. The control system 400 and/or the memory 300 can be centralized (within one housing) or decentralized (within two or more physically distinct housings). The control system 400′ and/or the memory 300′ can be centralized (within one housing) or decentralized (within two or more physically distinct housings).

In some implementations of the present disclosure, the processor 410 (or 410′ = in FIG. 1B) is configured to execute the machine-readable instructions 320 (or 320′ in FIG. 1B) to receive at least a portion of the data stored in the data repository 200 or 200′ (in FIG. 1B). In some such implementations, the portion of the data received corresponds to identifiable individuals. The first and second patient identification algorithms in memory 300 or 300′ (in FIG. 1B) process the received data, or a portion thereof, to determine preferred (e.g., likely to adhere long-term to treatment) identifiable individuals for OSA treatment.

In some implementations, a determined likelihood of obstructive sleep apnea for an individual to be identified in the initial group of individuals includes individuals meeting or exceeding a first threshold criteria (e.g., above 95% likelihood of OSA, above 90% likelihood of OSA, above 80% likelihood of OSA, above 70% likelihood of OSA, above 60% likelihood of OSA). In some implementations, a determined likelihood of long-term adherence to OSA treatment for an individual (for inclusion in a narrower subgroup of individuals) includes individuals associated with data meeting or exceeding a second threshold criteria (e.g., above 95% likelihood of adherence, above 90% likelihood of adherence, above 80% likelihood of adherence, above 70% likelihood of adherence, above 60% likelihood of adherence).

In some implementations, the processor 410 or 410′ executes machine-readable instructions 320 or 320′ to generate personalized treatment pathway(s) for one or more individuals in the narrower subgroup of preferred individuals for OSA treatment. The personalized treatment pathway is based on the physical, health, and/or behavioural characteristics data corresponding to each of the one of more individuals within the narrower subgroup.

It is contemplated that the systems described herein include identifying patients via an algorithm-driven module that have a threshold likelihood to have OSA and a threshold likelihood for long-term adherence to OSA treatment. The described systems and methods are desirable in the ability to review historical patient data to identify prior patients of a healthcare provider (e.g., cardiologist, endocrinologist, family practitioner), and based on the data, being able to identify individuals within those historical patient likely to have OSA and likely to adhere to a treatment plan.

In some implementations, the systems and methods can further direct a provider to a desired treatment pathway that will be successful for the identified patient. In the example of a cardiology healthcare provider, a historical patient with heart issues, or a historical patient on a path leading to heart issues, may be identified by the system as having a likelihood of OSA, which likely contributes to the heart issues. If the identified patient also has behavioral characteristics suggesting a likelihood of adherence to treatment of the OSA, patient information can then be sent to a healthcare provider, healthcare payor, or an integrated delivery network to allow this designated entity to consult with the historical patient.

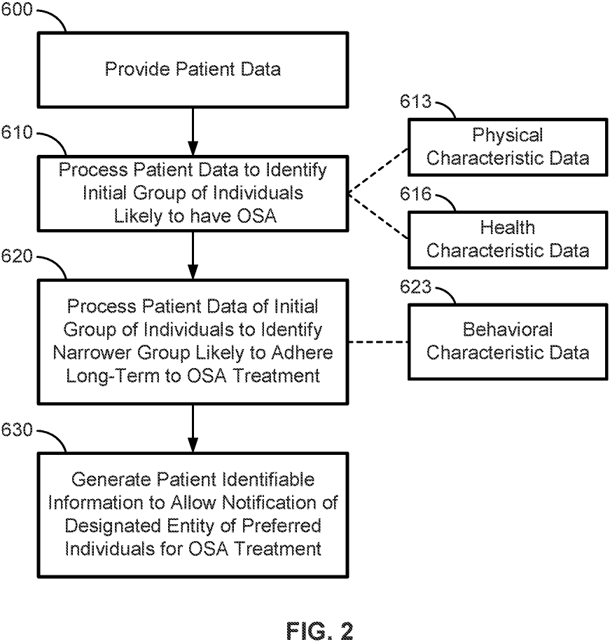

Referring now to FIG. 2, a process flow diagram is depicted of a method for identifying individuals with sleeping disorders and individuals having a long-term disposition to adopt a treatment plan. At step 600, patient data is provided that is stored or retrieved from a data repository. The patient data includes physical, health, and behavioral characteristic data corresponding to identifiable individuals. At step 610, a first patient identification algorithm is applied to process at least a portion of the patient data to identify an initial group of individuals associated with select physical and health characteristics. The physical and health characteristics may be derived from physical characteristic data 613 and health characteristic data 616. The identification of the initial group of individuals is based on a determined likelihood of obstructive sleep apnea for identifiable individuals meeting or exceeding a first threshold criteria. At step 620, a second patient identification algorithm is applied to process at least a portion of the patient data associated with the initial group of individuals to identify a narrower subgroup of individuals associated with select behavioral characteristics. The behavioral characteristics may be derived from behavioral characteristic data 623. The identification of the narrower group of individuals is based on a determined likelihood of long-term adherence to obstructive sleep apnea treatment for individuals in the narrower subgroup meeting or exceeding a second threshold criteria. At step 630, patient identifiable information from the patient data is generated to allow for notification of one or more designated entities that one or more of the individuals in the narrower subgroup are preferred individuals for obstructive sleep apnea treatment.

In some implementations, one or more designated entities can be notified, and may include a health care provider, an integrate delivery network, a health care payor, an administrator, at least one of the one or more individuals, or any combinations thereof.

In some implementations, personalized treatment pathways are generated for the one or more identified individuals based at least in part on the physical, health, and behavioral data corresponding to each of the one or more individuals within the narrower subgroup of individuals. For example, a personalized treatment pathway may include identifying a preferred method of sleep testing for an identified individual or an analysis of improved health outcomes by treating OSA. The improved health outcomes can include decreased mortality rate, readmits, hospital time, or any combinations thereof. Other improved health outcomes can include improved clinical, financial, and patient experiences. The generated personal treatment pathway may be transmitted to the corresponding individual, healthcare provider, other designated entity, or any combinations thereof.

In some implementation, a notification can include an analysis of potential healthcare cost savings by treating potential obstructive sleep apnea.

In some implementations, an alert is transmitted directly to a corresponding individual to inquire about a sleep test with their healthcare provider.

In some implementations, the generated patient identifiable information may be provided on a network server accessible to a third-party.

In some implementations, a data repository can include data associated with a care management platform, healthcare system, or both. In some implementations, patient data includes historical patient data.

In some implementations, one or more of the select physical and health characteristics include information provided by the identifiable individuals. In some implementations, the select health, behavioral, or demographic information is data input by a healthcare provider during one or more previous patient encounters.

In some implementations, a notification to an identified individual includes a direct message or an email message delivered through a health portal. In some implementations, a notification to a healthcare provider or administrator associated with the identified individuals includes an indication of the communication method most likely to result in patient follow up. In some implementations, the communication method includes one of a text message, email, phone call, or invitation to schedule a visit. In some implementations, the communication method may further include the delivery of the text message, email, phone call, or invitation to schedule a visit being initiated by one of an administrator, nurse, or physician.

In some implementations, a list of multiple individuals identified within the narrower subgroup of individuals is generated to direct proactive outreach.

In some implementations, the systems and methods include identifying missing patient data that would increase the accuracy of the identification of an individual for targeted follow up.

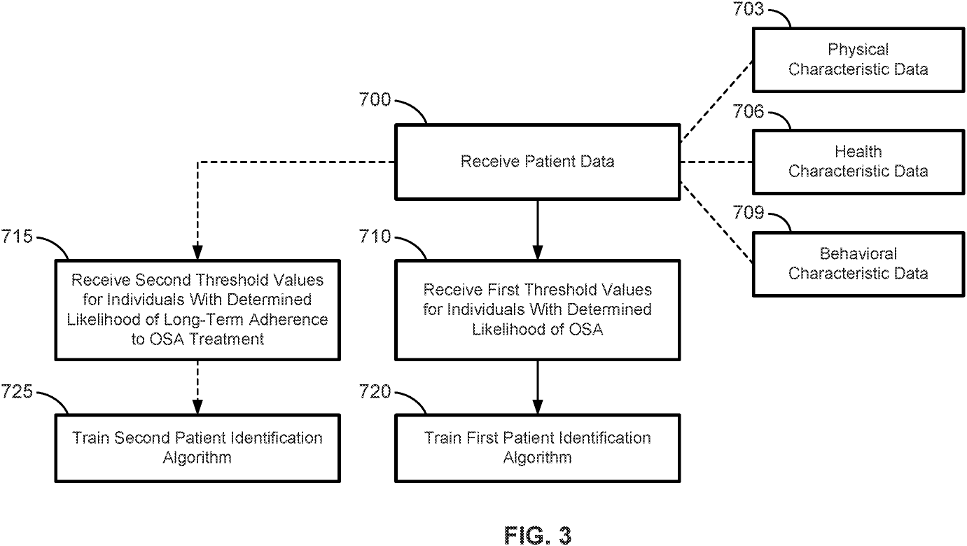

Referring now to FIG. 3, a process flow diagram is depicted of an exemplary method for training algorithms for identifying individuals with sleeping disorders and having a long-term disposition to adopt a treatment plan. At step 700, patient data is received and can include physical characteristic data 703, health characteristic data 706, and/or behavioral characteristic data 709. At step 710, first threshold values for identifiable individuals within the training patient data are determined, or received, and may include patient data associated with individuals known to have OSA. Next, at step 720, the first patient identification algorithm can be trained for identifying individuals based on a determined threshold for having likelihood of OSA. Similarly, at step 715, second threshold values for identifiable individuals within the training patient data are determined, or received, and may include patient data associated with individuals known to have long-term adherence to OSA treatment. Next, at step 720, the second patient identification algorithm can be trained for identifying individuals based on a determined threshold for long-term adherence to OSA treatment.

One or more elements or aspects or steps, or any portion(s) thereof, from one or more of any of claims 1 to 29 below can be combined with one or more elements or aspects or steps, or any portion(s) thereof, from one or more of any of the other claims 1 to 29 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method comprising:
providing patient data stored in a data repository, the patient data including physical, health, and behavioral data corresponding to identifiable individuals;
applying a first patient identification algorithm to process at least a portion of the patient data to identify an initial group of individuals associated with select physical and health characteristics, the identification of the initial group of individuals based on a determined likelihood of obstructive sleep apnea for identifiable individuals meeting or exceeding a first threshold criteria;
applying a second patient identification algorithm to process at least a portion of the patient data associated with the initial group of individuals to identify a narrower subgroup of individuals associated with select behavioral characteristics, the identification of the narrower group of individuals based on a determined likelihood of long-term adherence to obstructive sleep apnea treatment for individuals in the narrower subgroup meeting or exceeding a second threshold criteria; and
generating patient identifiable information from the patient data to allow for notification of one or more designated entities that one or more of the individuals in the narrower subgroup are preferred individuals for obstructive sleep apnea treatment.

2. The method of claim 1, wherein the one or more designated entities include a health care provider, an integrate delivery network, a health care payor, an administrator, at least one of the one or more individuals, or any combinations thereof.

3. The method of claim 1, further comprising generating personalized treatment pathways for the one or more individuals based at least in part on the physical, health, and behavioral data corresponding to each of the one or more individuals within the narrower subgroup of individuals.

4. The method of claim 3, further comprising transmitting a generated personal treatment pathway to a corresponding individual, healthcare provider, other designated entity, or any combinations thereof.

5. The method of claim 1, wherein the notification includes an analysis of potential healthcare cost savings by treating potential obstructive sleep apnea.

6. The method of claim 3, wherein the generated personal treatment pathway further includes an analysis of improved health outcomes by treating obstructive sleep apnea.

7. The method of claim 6, wherein the improved health outcomes include decreased mortality rate, readmits, hospital time, or any combinations thereof.

8. The method of claim 1, further comprising transmitting an alert directly to a corresponding individual to inquire about a sleep test with their healthcare provider.

9. The method of claim 3, wherein the generated personal treatment pathway includes a recommended sleep test method.

10. The method of claim 1, further comprising providing the generated patient identifiable information on a network server accessible to a third-party.

11-12. (canceled)

13. The method of claim 1, wherein one or more of the select physical and health characteristics are indirectly attributable to or intensified by obstructive sleep apnea.

14. The method of claim 1, wherein the select physical characteristics include neck circumference, weight, gender, blood pressure, age, body mass index, or any combinations thereof.

15. The method of claim 1, wherein one or more of the select physical and health characteristics include information provided by the identifiable individuals.

16. The method of claim 1, wherein the select health characteristics include snoring history, heart conditions, history of tiredness, observed apnea, diabetes, or any combinations thereof.

17. The method of claim 1, wherein the behavioral characteristics include demographic information, wherein the demographic information includes education, employment, place of residence, marital status, or any combinations thereof.

18. (canceled)

19. The method of claim 1, wherein the behavioral characteristics includes motivation, fitness level, exercise routine, adherence to prescribed medication protocols, adherence to prior doctor recommendations, or any combinations thereof.

20. The method of claim 1, wherein any of the select health, behavioral, or demographic information is data input by a healthcare provider during one or more previous patient encounters.

21. The method of claim 1, wherein a notification to an identified individual includes a direct message or an email message delivered through a health portal.

22. The method of claim 1, wherein a notification to a healthcare provider or administrator associated with the identified individuals includes an indication of the communication method most likely to result in patient follow up.

23-29. (canceled)

30. A system comprising:
a control system including one or more processors; and
a memory having stored thereon machine readable instructions such that when the machine executable instructions are executed by at least one of the one or more processors the system is configured to:
provide patient data stored in a data repository, the patient data including physical, health, and behavioral data corresponding to identifiable individuals;
apply a first patient identification algorithm to process at least a portion of the patient data to identify an initial group of individuals associated with select physical and health characteristics, the identification of the initial group of individuals based on a determined likelihood of obstructive sleep apnea for identifiable individuals meeting or exceeding a first threshold criteria;
apply a second patient identification algorithm to process at least a portion of the patient data associated with the initial group of individuals to identify a narrower subgroup of individuals associated with select behavioral characteristics, the identification of the narrower group of individuals based on a determined likelihood of long-term adherence to obstructive sleep apnea treatment for individuals in the narrower subgroup meeting or exceeding a second threshold criteria; and
generate patient identifiable information from the patient data to allow for notification of one or more designated entities that one or more of the individuals in the narrower subgroup are preferred individuals for obstructive sleep apnea treatment.
